(19) United States
(12) Patent Application Publication
(10) Pub. No.: US 2013/0337535 A1
(43) Pub. Date: Dec. 19, 2013

(54) Control of Cyanate in Aqueous Urea Solutions by Non-1,2-Ethylene Diamine Like Compounds for the Protection of Protein/Peptide Carbamylation

(71) Applicant: Fujifilm Diosynth Biotechnologies U.S.A., Inc., Morrisville, NC (US)

(72) Inventors: Philip A. Ropp, Chapel Hill, NC (US); Christie Lynn Williams, Holly Spring, NC (US); Michael Murray, Chapel Hill, NC (US); Miao Fang Lin, Cary, NC (US)

(73) Assignee: Fujifilm Diosynth Biotechnologies U.S.A., Inc., Morrisville, NC (US)

(21) Appl. No.: 13/971,423

(22) Filed: Aug. 20, 2013

Related U.S. Application Data
(62) Division of application No. 11/844,600, filed on Aug. 24, 2007.
Application No. 11/844,600 is a continuation of application No. 10/836,879, filed on Apr. 30, 2004, now abandoned.
Application No. 10/836,879 is a continuation-in-part of application No. 10/785,369, filed on Feb. 23, 2004, now Pat. No. 7,459,425.
(60) Provisional application No. 60/466,686, filed on Apr. 30, 2003.
(60) Provisional application No. 60/449,091, filed on Feb. 21, 2003.

(51) Int. Cl. C12N 9/96

(52) U.S. Cl. CPC C12N 9/96
USPC 435/188; 530/412

(57) ABSTRACT

Embodiments of the present invention generally relate to processing of peptides in urea solutions and substantial prevention of carbamylation of the peptide.

### CROSS REFERENCE TO RELATED APPLICATION

This application is a continuation of U.S. application Ser. No. 10/836,879 filed Apr. 30, 2004, which claims priority based on U.S. provisional application 60/466,686, filed Apr. 30, 2003, and a continuation-in-part of U.S. application Ser. No. 10/785,369, filed Feb. 23, 2004, which claims priority based on U.S. provisional application 60/449,091, filed on Feb. 21, 2003.

### FIELD OF THE INVENTION

The present invention is generally related to the control of cyanate in aqueous urea solutions used in the processing of proteins/peptides.

### BACKGROUND OF THE INVENTION

Urea containing solutions are commonly used to denature or solubilize proteins during protein purification/isolation processes and some analytical methods. One of the disadvantages to the use of urea solutions is the spontaneous formation of cyanate, from the urea, that can react with and modify proteins (Dirnhuber and Schütz, 1948). In urea containing solutions, cyanate reaches equilibrium with urea according to the following reaction:

H₂N—CO—NH₂ [symbol] CNO⁻+H⁺+NH₃⁻

The equilibrium concentration of cyanate in aqueous molar solutions of urea is dependent on the temperature and pH of the solution but has been shown to reach millimolar concentrations (Dirnhuber and Schütz, 1948; Marier and Rose, 1964; Hagel et al., 1971). The reactions of proteins and amino acid side chains with cyanate to yield carbamylated derivatives have been well characterized [reviewed in G. R. Stark, (1967)]. Cyanate can react with many protein functional groups (amino, sulfhydryl, carboxyl, hydroxyl, imidazole, and phosphate) but it is the reaction of cyanate with amino groups that are of primary concern. The carbamylation of amine groups is basically irreversible and leads to a change in the charge of the molecule. Carbamylated derivatives may have biological and antigenic properties that are different from those of the non-carbamylated molecules.

Protein carbamylation is a major issue both in vivo and in vitro. Lippincott and Apostol (1999) have shown that hemoglobins can be carbamylated on cysteines as an artifact of protein proteolytic digestion in the presence of urea. While Oimomi et al. (1987) demonstrated that carbamylated insulin had altered immunological and biological activities. Hasuike et al. (2002) have shown that in vivo cyanate can induce hemolysis by carbamylation of erythrocytes. Thus, carbamylated hemoglobin serves as a marker of posttranslational protein modification associated with uremic complications such as atherosclerosis. While Crompton et al. (1985) showed that the carbamylation of lens proteins by cyanate causes conformational changes that lead to cataracts.

Different methods to prevent protein carbamylation in vitro have been proposed. Lowering the solution temperature slows down both the cyanate formation and subsequent carbamylation, but increases solution viscosity, which can impact downstream processes such as filtration and chromatography. Deionization of urea solutions only temporarily removes cyanate from urea solutions and usually this can not be done in the presence of proteins. Lowering the solution pH to 2 decreases cyanate formation and causes the decomposition of cyanate to carbon dioxide and ammonia but is unsuitable for most proteins. Amine-specific derivatization and deprotection of proteins is not a convenient quantitative approach. Although these approaches have applications in special circumstance, none can be generally applied in the field.

Since cyanate formation in the urea buffer cannot be prevented under the condition of normal protein purification, an alternative approach would be to remove cyanate as it forms by the use of cyanate scavengers. A search for cyanate scavengers has been reported (DiMarchi, 1986). This work identified 1,2-ethylene diamine like compounds as scavengers based on their ability to protect insulin from carbamylation. In addition, these compounds are relatively inexpensive, inert, soluble, and readily removable. The DiMarchi process defines a 1,2-ethylene diamine like compound as:

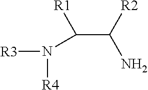

Where R₁, R₂, R₃, and R₄ are groups that, taken together, do not significantly alter the amino group pKa values or the steric accessibility of the amino groups relative to the 1,2-ethylene diamine itself. DiMarchi stresses that it is the steric arrangement of the 1,2-ethylene diamine like compound that provides for the scavenging ability.

However, while the 1,2-ethylene diamine like compounds possess good cyanate scavenging ability, they are highly basic and strongly influence pH and buffering capacity when used at the concentration suggested by DiMarchi. Therefore the artfield is in search of other compounds and/or groups of compounds that function as carbamylation scavengers without the disadvantages of the 1,2-ethylene diamine like compounds. Such compounds should either be more effective scavengers than 1,2-etylene diamine, so that they can be used in sub-millimolar concentration, or significantly less basic than 1,2-ethylene diamine, preferably with low or no net charge at the experimental conditions, having low impact on the buffering capacity of typical biological buffers when used at a millimolar concentrations. Alternatively, such compounds would have buffering capacities within or close to the neutral range and could be used as buffers agents themselves.

Other problems experienced in the protein purification art include the short shelf life of urea containing reagents because of the decomposition of the urea. Such problems are addressed in many product manuals. For example, Novagen markets a “HIS-BIND Kit” for the purification of proteins containing a His-Tag sequence by metal chelation chromatography. (HIS-BIND Kits, Novagen, TB054 Rev.C 1102, 2002, p. 2). On page 15 of the HIS-BIND Kit instructions, a cautionary note is made that the urea solution used must be made fresh and used promptly because urea decomposes to form cyanate ions, which can covalently modify primary amines on target proteins. Similar warnings and notices are found on the September 2002 revision of the Ni—NTA Superflow BioRobot Handbook. On page 52 of the Superflow BioRobot Handbook, under Protocol for the BioRobot 3000, Reference Numeral #1, a caution is given that due to the dissociation of urea, the pH of the buffers should be adjusted immediately before use. Accordingly, the artfield is in search of a process of preparing and/or storing process solutions for the purification of proteins whereby carbamylation of the proteins in a urea buffer is substantially inhibited or delayed.

### SUMMARY OF THE INVENTION

Embodiments of the present invention generally relate to processes utilizing a class of non-1,2-ethylene diamine like compounds that are capable of substantially inhibiting and/or delaying carbamylation of peptides in process solutions. In an embodiment, the process solution is in a urea buffer.

Using bovine pancreatic ribonuclease (RNase A) as a model protein, it has been found that several non-1,2-ethylene diamine like compounds, such as glycinamide, histidine, 4-hydroxy proline, and some dipeptides, such as Glycyl-Glycine, and Glycyl-Histidine, significantly inhibited carbamylation of RNase A. Unexpectedly, these compounds are not 1,2-ethylene diamine like compounds and are not expected to act as carbamylation inhibitors as defined in the DiMarchi process.

Further studies illustrate that the above non-1,2-ethylene diamine like compounds and others may be used as additives to urea containing process solutions to increase their shelf life by preventing the accumulation of cyanate in solutions.

### DETAILED DESCRIPTION OF THE INVENTION

As used herein, the term “1,2-ethylene diamine like compounds” means and refers to a compound structurally related to, or like, 1,2-ethylene diamine, as described by DiMarchi, and having some carbamylation inhibition and/or reduction characteristics similar thereto.

The processing of peptides as contemplated herein encompasses any of a wide range of peptide processing. Typical, non-limiting, examples are purification, chemical modification, including, e.g., peptide sulfitolysis, and other such peptide processing steps.

Accordingly, in an embodiment, the present invention comprises a process for inhibiting and/or delaying carbamylation of a peptide/protein in a urea containing solutions during processing of said peptide/protein comprising the step of adding a carbamylation inhibiting compound to the process wherein said compound is not a 1,2-ethylene diamine like compound.

In an embodiment, the compound is selected from the group consisting of glycinamide, histidine, 4-hydroxy proline, Glycyl-Glycine, and Glycyl-Histidine.

Generally, the concentration of the scavenger compound used in the process of this invention is within the range from about 1 mM to about 500 mM. In an embodiment, the concentration of the compound is within the range from about 10 mM to about 100 mM, based upon the total processing medium. In another embodiment, the concentration of the compound is within the range from about 25 mM to about 50 mM. However, the concentration of the compound may vary according to the concentration of the cyanate in solution.

Carbamylation inhibition during peptide/protein processing is available for essentially any peptide and/or protein, irrespective of structure, when subjected to conditions in which amounts of cyanic acid can be expected to be present. Thus, for example, and not by way of limitation, peptides/proteins such as ribonucleases, insulin A-chain, insulin B-chain, proinsulin, C-peptide, pancreatic polypeptide, growth hormone, growth hormone releasing factor, insulin-like growth factor, somatostatin, and, others are suitable for use with the novel non-1,2-ethylene diamine like compounds of the present invention. Preferred peptides/proteins are soluble in urea and readily carbamylated in the presence of cyanate.

The compounds described in this invention do not possess the diamine functionality characteristic of 1,2-ethylene diamine-like compounds described by DiMarchi, and In an embodiment, the compound is selected from the group consisting of glycinamide, histidine, 4-hydroxy proline, Glycyl-Glycine, and Glycyl-Histidine. Surprisingly, the compounds selected from this group show comparable cyanate-scavenging and carbamylation-protecting properties to 1,2-ethylene diamine while lacking the diamine functionality. Unexpectedly, it has been further observed, that some of the cyanate scavenging compounds described in the current invention do not possess any primary amine or sulfhydryl functionality while still showing the ability to scavenge cyanate and protect against carbamylation, the examples being diethanolamine and 4-hydroxy proline.

Further unexpectedly, it has been found that it is not necessary or required that the compound by sterically unhindered as proposed by DiMarchi for the compound to function as a cyanate scavenger. Compounds selected from a group of non-1,2-ethylene diamine like compounds that varied sterical constrains around the amino group inhibit and/or delay carbamylation of peptides/proteins with comparable results. In an embodiment, the compound is selected from the group consisting of glycinamide, histidine, 4-hydroxy proline, Glycyl-Glycine, and Glycyl-Histidine.

The structures of the compounds are as follows:

| | | Approximate pKa | | |
|---|---|---|---|---|
| Compound | Structure | pK₁ | pK₂ | pK₃ |
| Glycinamide | 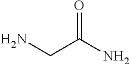 | 8.2 | | |
| Histidine | 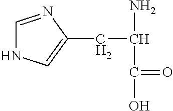 [SMILES: NC(Cc1c[nH]cn1)C(=O)O] | 1.82 | 6.00 | 9.17 |
| 4-Hydroxy Proline | 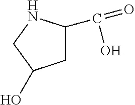 | 1.92 | 9.73 | |
| Glycyl- Glycine | 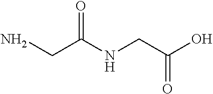 | 3.12 | 8.17 | |
| Diethanolamine | 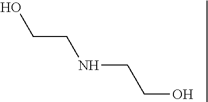 | 8.88 | | |
| Glycyl- Histidine | 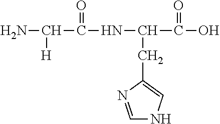 [SMILES: NCC(=O)NC(Cc1c[nH]cn1)C(=O)O] | 3.1 | 6.77 | 8.25 |

As can be seen, when compared to ethylene diamine like compounds,

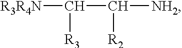

selected compounds of the present invention, such as 4-hydroxy proline or diethanolamine containing secondary amine groups, cannot be referred to as sterically unhindered. Therefore, it is proposed that the effectiveness of the compounds of the present invention is determined by the stability of a cyanate-scavenger adduct rather than any sterical constrains within the scavenger itself. Such compounds include but are not limited to 4-hydroxy proline, histidine, histidyl-glycine, and diethanolamine, consequently, the effectiveness of the compounds described in the present invention can not be predicted from the work described by DiMarchi.

In another surprising fact, the pKa value(s) of the amino group of a compound may vary significantly from the pKa of a 1,2-ethylene diamine like compounds while still retaining a good ability to inhibit and/or delay the carbamylation of peptides/protein during processing. DiMarchi proposed that 1,2-ethylene diamine like compounds provide superior protection of proteins to carbamylation because the pKa values of 1,2-ethylene diamine (7.5 and 10.7), and like compounds, are very close to the pKa values of the N-terminal and lysine side chain amino groups (8.0 and 10.0, respectively). In various embodiments of the present invention an amine may be used with a pKa of about 8.20. Notably, such a mono-amine compounds would not be predicted to function as carbamylation inhibitors and/or delayors, a suitable example being glycinamide and/or glycine-glycine. In another embodiment, three groups having varying pKa values of about 1.82, about 6.0 and about 9.17, a suitable example being Histidine with a —COOH, a —NH₂, and a side chain. In another embodiment, two groups with pKa values of about 1.92 and about 9.73, a suitable example being hydroxy-proline.

As defined above, embodiments of the present invention encompass numerous processes to which peptides/proteins are subjected. In an embodiment, the process is solubilizing the peptide/protein in urea. In another embodiment, the process is purification of peptide/protein. In another embodiment, the process is extending shelf life of urea containing solutions. However, the invention may comprise other processes.

For example, general embodiments comprise both processes and solutions, without limitation. Embodiments of the present invention generally comprise a storable urea-based peptide processing solution comprising a urea-based peptide processing solution containing a sufficient quantity of a non-1,2-ethylene diamine like compound to maintain cyanate concentration in the solution at levels to prevent substantial carbamylation of the peptide during processing. In further embodiments, the concentration of the non-1,2-ethylene diamine like compound is between about 1 mM and about 150 mM. In further embodiments, the pH of the solution is between about 4.5 and about 8.5. In other embodiments, the non-1,2-ethylene diamine like compound is selected from the group consisting of L-Glycine, Diethanolamine, L-Histidine, L-Arginine, L-Threonine, L-Lysine, L-Cysteine, Taurine, Hydralazine, Histidyl-Glycine, 4-Hydroxy-Proline, Glycyl-Glycine, Glycinamide, and Tri-Glycine.

Embodiments of processes of the present invention include a process for the preparation of a storable urea-based peptide processing solution for use in a peptide process comprising the step of adding a sufficient quantity of a non-1,2-ethylene diamine like compound to the processing solution to maintain cyanate concentration in the solution at levels to prevent substantial carbamylation of the peptide during processing. Further embodiments comprise a peptide process that is chosen from the group selected from purification, chemical modification, and peptide sulfitolysis. As well, embodiments of the process further comprise storing the solution for up to 35 days. Other embodiments contemplate storage for periods exceeding 35 days.

Various embodiments of the present invention inhibit carbamylation of the peptide/protein to varying degrees. In an embodiment, the carbamylation percent protection is about 100% after three weeks. In another embodiment, a compound of the present invention inhibits carbamylation of ribonuclease A to a greater extent than does 1,2-ethylene diamine inhibit the carbamylation of ribonuclease A. Preferred compounds for comparison comprise a compound selected from the group consisting of histidine, 4-hydroxy proline, and Glycyl-Glycine.

While the invention has been described in connection with specific embodiments thereof, it will be understood that it is capable of further modifications and the appended. Claims are intended to cover any variations, uses, or adaptations of the invention following, in general, the principles of the invention and including such departures from the present disclosure as come within known or customary practice within the art to which the invention pertains and as may be applied to the essential features herein before set forth whether now existing or after arising. Further, while embodiments of the invention have been described with specific dimensional characteristics and/or measurements, it will be understood that the embodiments are capable of different dimensional characteristics and/or measurements without departing from the principles of the invention and the appended Claims are intended to cover such differences. Furthermore, all patents and other publications mentioned herein are herby incorporated by reference.

### EXAMPLES

In this experiment, a series of amines, amides, amino acids, and di- and tri-peptides were tested and compared with 1,2-ethylene diamine, and their efficiency quantified as percent protection against RNase A carbamylation. In our study on cyanate scavengers for protein carbamylation protection, bovine pancreas RNase A (124 amino acid residues, ˜14 kDa) served as the model protein. It is a notably stable enzyme that is inactivated by long exposure at moderate temperature to urea (CNO⁻) by chemical changes at the 8 Cys, 10 Lys, 4 Arg, 4 His residues.

Materials and Methods

Cyanate Carbamylation Study

A 2 mg/ml stock solution of bovine RNase A (Sigma), a 100 mM stock solution of sodium cyanate (Sigma), and a stock solution of 250 mM sodium phosphate pH 7.9 were prepared in water and stored at −70° C. The compounds to be tested (Table 1) were prepared in 0.5 M stock concentrations, adjusted to pH 8 using HCl or NaOH, and stored at room temperature. The pH of the histidine solution was not adjusted (it could not be adjusted properly). Due to solubility limitation, a 0.2 M stock solution was made for Tri-Glycine. All amino acids were the L isomer.

RNase A, at 1 mg/ml, was carbamylated by incubation with cyanate at room temperature. Different scavenger concentrations (100, 50, 10 and 5 mM) were tested for protective potential. The final concentrations of other components in the carbamylation reaction mixture were 5 mM for CNO and 50 mM sodium phosphate pH 7.9.

The controls were added to each reaction setup: A negative control, which had neither cyanate nor scavenger, was used for quantifying the RNase natural decay. A positive control, which had 5 mM cyanate, but no any scavenger reagent, was used for estimating the completion of the carbamylation reaction.

In all experiments, 5 mM cyanate was added last.

Non-carbamylated RNase A was quantitatively determined by HPLC on a Mono S column (Amersham Biosciences, Piscateway, N.J.). Aliquots of the carbamylation reaction mixture were taken at 0, 3, 7, 14, and 21 day time points and the samples were analyzed by HPLC. If not used immediately, the samples were frozen at −70° C. Prior to HPLC, the samples were titrated to pH 5 using 38% acetic acid solution to a final 1:1 dilution. A buffer system consisting of 50 mM ammonium acetate pH 5 (buffer A) and 1 M ammonium acetate pH 5 (buffer B) was used for separation of carbamylated and non-carbamylated RNase species. A gradient was used from 10% B to 70% B over 14 min. Then the column was washed with 70% buffer B for 2 min. At 16 min the mobile phase was switched back to 10% B to equilibrate the system before next injection. All chromatographic separations were carried out at 10° C. using a mobile phase flow rate of 1 ml/min and 100 μl sample injection volume.

The amount of remaining non-carbamylated RNase A in the test was converted to percent carbamylation protection. To account for differences in samples, time 0 of each tested group was considered to be 100% protection based on the assumption that there is no carbamylation at time point 0. The data were further corrected point-by-point for natural protein decay.

Cyanate Assay

To check for CNO-/scavenger interaction and estimate reaction kinetics, the same reaction setup was used, but no protein was added to final mixture. Aliquots were taken at 2 h and 24 h time points and treated as above. A modified HPLC procedure (Black & Schulz, 1999) was used for free cyanate detection. The HPLC samples were diluted 1:20 with water. Separations were carried out at room temperature, using a mobile phase flow rate of 1.2 ml/min and 100 μl injection volume. The results were normalized to 5 mM cyanate, the starting cyanate concentration. In some cases, the scavenger peak overlaps the cyanate peak, so the integration values are smaller than expected.

Urea Carbamylation Assay

A 9 M urea (JT Baker) stock solution was made fresh in 50 mM sodium phosphate, pH 7.9, and used immediately to make up a 1.1 mg/ml RNase A stock solution. The final urea concentration in the carbamylation mixture was 8.1 M and the RNase A was 1 mg/ml. No sodium cyanate added to the reaction mixture. The experiment further proceeded as described in cyanate carbamylation study.

Results and Discussion

Cyanate Carbamylation Study

Table 1 summarizes the initial compound screen for their potential protection against RNase A carbamylation by cyanate. Only those compounds, tested at 100 mM concentration, that provide greater than 70% protection against carbamylation by 5 mM cyanate at the end of 3 weeks are listed. Five compounds, in addition to 1,2-ethylene diamine, displayed a greater than 90% protection level. These compounds were glycinamide, L-Histidine, 4-hydroxy-Proline, glycyl-glycine, and glycyl-histidine.

TABLE 1
Tested compounds in the carbamylation experiment. The percent
protection was determined for 100 mM scavenger after
3 weeks in the presence of 5 mM cyanate.

| | Carbamylation | Approximate pKa | | |
|---|---|---|---|---|
| Tested Compound | Protection (%) | pK₁ | pK₂ | pK₃ |
| L-Histidine | 100 | 1.82 | 6.00 | 9.17 |
| 4-Hydroxy-Proline | 100 | 1.92 | 9.73 | |
| Glycyl-Glycine | 100 | 3.12 | 8.17 | |
| 1,2-Ethylene diamine | 99 | 7.5 | 10.7 | |
| Glycyl-Histidine | 95 | 3.1 | 6.77 | 8.25 |
| Glycinamide | 93 | 8.2 | | |
| Histidyl-Glycine | 85 | 3.1 | 5.85 | 7.69 |
| Diethanolamine | 82 | 9.0 | | |
| L-Glycine | 77 | 2.34 | 9.6 | |
| L-Lysine | 77 | 2.18 | 8.95 | 10.53 |
| L-Arginine | 77 | 2.17 | 9.04 | 12.48 |
| Tri-Glycine | 77 | 3.26 | 7.91 | |

The time and concentration dependence of these compounds for the protection of RNase A against carbamylation by 5 mM cyanate is shown in Table 2. The positive control was set with RNase A, 5 mM cyanate, but without any potential protection reagents. The negative control was only the RNase A in test buffer. Data from Table 2 clearly proved that the carbamylation of RNase A by cyanate was inhibited with 50 or 100 mM tested reagents, compared to the positive control.

TABLE 2
Selected results of RNase cyanate carbamylation study.

| Tested compounds and | | Carbamylation Protection (%) | | | | |
|---|---|---|---|---|---|---|
| concentration | | Day 0 | Day 3 | Day 7 | Day 14 | Day 21 |
| 1,2-Ethylene | 100 mM | 100 | 98 | 96 | 96 | 93 |
| diamine | 50 mM | 100 | 94 | 93 | 93 | 90 |
| L-Histidine | 100 mM | 100 | 93 | 97 | 102 | 102 |
| | 50 mM | 100 | 68 | 70 | 76 | 77 |
| Glycinamide | 100 mM | 100 | 97 | 96 | 94 | 93 |
| | 50 mM | 100 | 93 | 92 | 92 | 87 |
| Glycyl- | 100 mM | 100 | 98 | 98 | 100 | 101 |
| Glycine | 50 mM | 100 | 91 | 92 | 94 | 93 |
| Glycyl- | 100 mM | 100 | 97 | 95 | 97 | 95 |
| Histidine | 50 mM | 100 | 95 | 95 | 96 | 91 |
| 4-Hydroxy- | 100 mM | 100 | 104 | 102 | 98 | 103 |
| Proline | 50 mM | 100 | 102 | 101 | 102 | 100 |
| Positive | | 100 | 36 | 16 | 11 | 11 |
| control | | | | | | |
| Negative | | 100 | 94 | 93 | 89 | 89 |
| control | | | | | | |

Cyanate Scavenging Study

In order to verify the mechanism of the carbamylation protection by the tested compounds, a residual cyanate level test was performed. Compounds, at the concentrations indicated, were mixed with 5 mM cyanate and the reaction mixtures were analyzed for cyanate after 2 and 24 hours. Table 3 shows the results of cyanate scavenging study. The residual cyanate was calculated based on the percent cyanate remaining from the starting concentration at 2 and 24 hour time points. All tested compounds showed over 50% cyanate scavenging capability after 24 hr at the concentration of 25 mM or greater, except the diethanolamine. At compound concentrations below 10 mM, the cyanate scavenging potential was not conclusive. The data agreed well with the results of cyanate carbamylation protection study on RNase A. Based on these results, the mechanism of the carbamylation protection on RNase A could be attributed to the cyanate scavenging.

TABLE 3
Results of cyanate scavenging.

| Tested | | CNO⁻ Remaining (%) | | | | |
|---|---|---|---|---|---|---|
| Reagents | Time | 100 mM | 50 mM | 25 mM | 10 mM | 5 mM |
| 1,2-Ethylene | 2 h | 20.0 | 45.0 | NA | 67.6 | 74.6 |
| diamine | 24 h | 0 | 2.5 | 16.4 | 43.8 | 87.8 |
| Diethanolamine | 2 h | 45.4 | 92.1 | 87.5 | 78.2 | 78.8 |
| | 24 h | 5.5 | 4.2 | 59.6 | 79.7 | 75.3 |
| Glycyl-Glycine | 2 h | 23.1 | 66.4 | 80.1 | 79.3 | 82.7 |
| | 24 h | 0 | 3.0 | 11.9 | 42.4 | 64.1 |
| L-Histidine | 2 h | 22.9 | 73.2 | 91.0 | 49.5 | 85.1 |
| | 24 h | 2.0 | 10.9 | 32.5 | 59.6 | 79.9 |
| Glycinamide | 2 h | 29.5 | 49.5 | 80.2 | 70.9 | 78.7 |
| | 24 h | 2.2 | 4.4 | 19.0 | 0.9 | 65.7 |
| Histidyl- | 2 h | 39.5 | 52.8 | 70.9 | 28.6 | 81.4 |
| Glycine | 24 h | 5.0 | 20.2 | 31.5 | 60.4 | 81.7 |
| Glycyl- | 2 h | 37.1 | 49.2 | 76.7 | 63.0 | 86.2 |
| Histidine | 24 h | 0.8 | 6.9 | 20.9 | 50.9 | 77.6 |
| Tri-Glycine | 2 h | 42.2 | 52.6 | 68.9 | 50.9 | 1.2 |
| | 24 h | 1.1 | 4.6 | 11.5 | 81.5 | 34.1 |
| 4-Hydroxy- | 2 h | 38.2 | 40.9 | 61.2 | 64.4 | 61.5 |
| Proline | 24 h | 1.1 | 8.5 | 26.6 | 57.2 | 72.2 |

Urea Carbamylation Study

Cyanate accumulation in urea buffers is a gradual process. To demonstrate that the inclusion of cyanate scavengers in urea buffers can inhibit protein carbamylation a urea carbamylation study was performed. In this experiment, urea in the process buffer was the source of cyanate responsible for the carbamylation of RNase A. The urea carbamylation study was set with 1 mg/ml RNase A in 8 M urea buffer, pH 7.9, containing different concentration of scavenger reagents over a period of three weeks. The results of this experiment are summarized in Table 4. The data showed that all scavengers tested were able to protect RNase A against carbamylation to some degree. The trend was the same as observed in cyanate carbamylation study however the degrees of protection observed were consistently lower than those observed from the direct cyanate carbamylation studies. There are several possible explanations for this discrepancy. The kinetics of carbamylation might be different in urea. RNase A is unfolded in urea so more sites are exposed for carbamylation. And/or in the urea system, cyanate is continually being formed from the decomposition of urea where as in the cyanate studies once the scavenger removes cyanate it is not replaced. The recommended scavenger concentration for preventing RNase A carbamylation is 25 mM or greater.

TABLE 4
Results of RNase A urea carbamylation study.

| Tested reagents | | Protection of carbamylation in urea (%) | | | | |
|---|---|---|---|---|---|---|
| And the concentration | | Day 0 | Day 3 | 1 week | 2 week | 3 week |
| 1,2-Ethylene | 50 mM | 100 | 83 | 62 | 65 | 54 |
| diamine | 25 mM | 100 | 88 | 63 | 61 | 53 |
| | 10 mM | 100 | 83 | 60 | 50 | 37 |
| L-Histidine | 50 mM | 100 | 101 | 98 | 91 | 80 |
| | 25 mM | 100 | 85 | 67 | 41 | 30 |
| | 10 mM | 100 | 84 | 43 | 22 | 16 |
| Glycinamide | 50 mM | 100 | 83 | 66 | 60 | 51 |
| | 25 mM | 100 | 85 | 64 | 56 | 46 |
| | 10 mM | 100 | 81 | 58 | 39 | 29 |
| Glycyl-glycine | 50 mM | 100 | 92 | 76 | 64 | 60 |
| | 25 mM | 100 | 88 | 71 | 56 | 50 |
| | 10 mM | 100 | 59 | 62 | 44 | 32 |
| Histidyl- | 50 mM | 100 | 88 | 67 | 66 | 63 |
| Glycine | 25 mM | 100 | 82 | 64 | 52 | 43 |
| | 10 mM | 100 | 82 | 51 | 38 | 27 |
| Glycyl- | 50 mM | 100 | 88 | 69 | 63 | 53 |
| Histidine | 25 mM | 100 | 86 | 61 | 49 | 46 |
| | 10 mM | 100 | 84 | 58 | 38 | 27 |
| Tri-Glycine | 50 mM | NA | NA | NA | NA | NA |
| | 25 mM | 100 | 85 | 62 | 49 | 43 |
| | 10 mM | 100 | 85 | 61 | 42 | 28 |
| Hydroxy- | 50 mM | 100 | 90 | 69 | 55 | 50 |
| proline | 25 mM | 100 | 86 | 53 | 47 | 39 |
| | 10 mM | 100 | 80 | 52 | 31 | 24 |
| Control | 0 mM | 100 | 86 | 33 | 13 | 11 |

Process Solution Storage Studies

In this experiment scavengers were added to a final concentration of 25 mM to 8 M urea solutions prepared in either 50 mM Tris, pH 8.0, or 50 mM HEPES, pH 7.0. The solutions were incubated at room temperature (17 to 20° C.) for up to 35 days. Cyanate concentrations were determined and compared to controls, 8 M urea in buffer without scavengers. The data in Table 5 showed that the tested 1,2-non-ethylene diamine like compounds could function to maintain low levels of cyanate in process solutions that require storage prior to use. Trends were similar at pH 7 and pH 8 although the efficiency of protection was slightly better at pH 7. In this manner, process solutions containing cyanate scavengers could be made up well before they are required for use, thereby maximizing protein processing time.

TABLE 5
Efficiency of 25 mM Cyanate Scavengers in 8M
urea buffers at pH 7.0 and 8.0

| | | | | | pH 8.0 | pH 7.0 |
|---|---|---|---|---|---|---|
| | | | | | t = 28-35 days | t = 30 days |
| | | | | | Residual | Residual |
| | | Approximate pKa | | | Cyanate | Cyanate |
| Tested Compounds | | pK₁ | pK₂ | pK₃ | (%) | (%) |
| Control | Tris: | 8.06 | | | 100 | 100 |
| | HEPES: | 7.55 | | | | |

| | | | | | |
|---|---|---|---|---|---|
| L-Glycine | 2.34 | 9.6 | | 24 | ND |
| Diethanolamine | 9.0 | | | 23 | ND |
| L-Histidine | 1.82 | 6.00 | 9.17 | 22 | 9 |
| L-Arginine | 2.17 | 9.04 | 12.48 | 21 | ND |
| L-Threonine | 2.09 | 9.10 | | 21 | ND |
| L-Lysine | 2.18 | 8.95 | 10.53 | 20 | ND |
| L-Cysteine | 1.96 | 8.18 | 10.28 | 20 | ND |
| Taurine | 1.5 | 8.74 | | 13 | 9 |
| Hydralazine | 2.53 | 7.17 | | 14 | 4 |
| Histidyl-Glycine | 3.1 | 5.85 | 7.69 | 12 | ND |
| 4-Hydroxy Proline | 1.92 | 9.73 | | 10 | ND |
| Glycyl-Glycine | 3.12 | 8.17 | | 8 | 5 |
| Glycinamide | 8.2 | | | 6 | 3 |
| Tri-Glycine | 3.26 | 7.91 | | 6 | ND |
| 1,2-Ethylene | 7.5 | 10.7 | | 4 | 2 |
| diamine | | | | | |

Conclusions

Some compounds have the potential to prevent protein carbamylation. Compounds, such as L-Histidine, glycinamide, 4-hydroxy-proline, Glycyl-Glycine, Glycyl-Histidine, and Histidyl-Glycine, as well as Tri-Glycine, afforded significant protection to RNase A and other proteins against carbamylation by cyanate, either directly added to the protein containing solutions or generated via the decomposition of urea. These compounds also posses the ability to extend the life of urea containing process solutions by preventing the accumulation of cyanate in the solutions. The protection of RNase A by the tested compounds is concentration dependent, with most compounds proficient at 25 mM or greater. RNase A in that about 20% of the amino acid residues of RNase A are susceptible to carbamylation, serves as an excellent model protein for this study. The concentration of scavenger could vary, depending on the available carbamylation sites of the target protein. Based on the data collected, the cyanate scavengers tested here can be used in protein purification processes and in process solutions.

### REFERENCES

Dirnhuber P., and Schütz F., Biochem. J. 1948, 42: 628-632. The isomeric transformation of urea into ammonium cyanate in aqueous solutions.
Stark, G. R., in: Methods in Enzymology, vol. 11, eds. S. P. Colowick and N. O. Kaplan. (Academic Press, New York, London, 1967) p. 590. Modification of proteins by cyanate.
Marier, J. R., and Rose, D. Anal. Biochem. 1964, 7: 304-314. Determination of cyanate, and a study of its accumulation in aqueous solutions of urea.
Hagel, P., Gerding, J. J. T., Fieggen, W., and Bloemendal, H. Biochim. Biophys. Acta, 1971, 243: 366-373. Cyanate formation in solutions of urea. I. Calculation of cyanate concentrations at different temperature and pH.
Crompton M, Ixon K C, Harding J J. Exp. Eye Res. 1985, 40: 297-311. Aspirin prevents carbamylation of soluble lens proteins and prevents cyanate-induced phase separation opacities in vitro: a possible mechanism by which aspirin could prevent cataract.
DiMarchi, R D UD Patent 4605513, 1986. Eli Lilly co. Process for inhibiting peptide carbamylation.
Hasuike Y, Nakanishi T, Maeda K, Tanaka T, Inoue T, Takamitsu Y. Nephron 2002, 91: 228-234. Carbamylated hemoglobin as a therapeutic marker in hemodialysis.
Lippincott J, Apostol I. Anal. Biochem. 1999, 267: 57-64. Carbamylation of cysteine: a potential artifact in peptide mapping of hemoglobins in the presence of urea.
Oimomi M, Hatanaka H, Yoshimura Y, Yokono K, Baba S, Taketomi Y. Nephron 1987, 46: 63-6. Carbamylation of insulin and its biological activity.
Black S B and Schulz R S. J. Chrom. A. 1999, 855: 267-272. Ion chromatography determination of cyanate in saline gold processing samples.

1-19. (canceled)

20. A method of inhibiting and/or delaying carbamylation of a polypeptide in a urea and/or cyanate containing solution, the method comprising a step of adding a carbamylation-inhibiting compound to the solution,
wherein said carbamylation-inhibiting compound is selected from the group consisting of L-Glycine, L-histidine, Diethanolamine, L-Arginine, L-Threonine, L-Lysine, L-Cysteine, Taurine, Hydralazine, 4-Hydroxy-Proline, a dipeptide and Tri-Glycine.

21. The method according to claim 20, wherein the polypeptide is a ribonuclease.

22. The method according to claim 21, wherein the ribonuclease is RNase A.

23. The method according to claim 20, wherein the carbamylation-inhibiting compound is added to the solution in an amount effective to provide about 100% carbamylation protection of the polypeptide for a period of three weeks.

24. The method according to claim 20, wherein the concentration of the carbamylation-inhibiting compound is between 1 mM and 150 mM.

25. The method according to claim 20, wherein the carbamylation-inhibiting compound has a buffering capacity of about neutral.

26. A method of inhibiting and/or delaying carbamylation of a polypeptide in a urea and/or cyanate containing solution, the method comprising a step of adding a carbamylation-inhibiting compound to the solution,
wherein the carbamylation-inhibiting compound is selected from the group consisting of L-Glycine, L-histidine, Diethanolamine, L-Arginine, L-Threonine, L-Lysine, L-Cysteine, Taurine, Hydralazine, 4-Hydroxy-Proline, Glycinamide, a dipeptide and Tri-Glycine

27. The method according to claim 26, wherein the carbamylation-inhibiting compound has a buffering capacity of about neutral.

28. A method of inhibiting and/or delaying carbamylation of a polypeptide in a urea and/or cyanate containing solution, the method comprising a step of adding a carbamylation-inhibiting compound selected from the group consisting of L-Glycine, Diethanolamine, L-Arginine, L-Threonine, L-Lysine, L-Cysteine, Taurine, Hydralazine, Glycinamide, a dipeptide and Tri-Glycine to the solution,
wherein the carbamylation-inhibiting compound is added to the solution in an amount effective to provide about 100% carbamylation protection of the polypeptide for a period of three weeks.

29. The method according to claim 28, wherein the concentration of the carbamylation-inhibiting compound is between 1 mM and 150 mM.

30. The method according to claim 29, wherein the cyanate in the solution is at a concentration of about 5 mM.
